(19) United States
(12) Patent Application Publication
(10) Pub. No.: US 2025/0245834 A1
(43) Pub. Date: Jul. 31, 2025

(54) SYSTEM AND METHODS FOR PREDICTING GLAUCOMA INCIDENCE AND PROGRESSION USING RETINAL PHOTOGRAPHS

(71) Applicants: Charlotte Zhang, Macau (MO); Yuanxu Gao, Macau (MO)

(72) Inventors: Charlotte ZHANG, San Diego, CA (US); Yuanxu GAO, Weifang (CN)

(21) Appl. No.: 18/870,188

(22) PCT Filed: May 31, 2023
(86) PCT No.: PCT/US2023/023908

§ 371 Date: Nov. 27, 2024

Related U.S. Application Data
(60) Provisional application No. 63/347,399, filed on May 31, 2022.

(51) Int. Cl. G06T 7/00; A61B 3/12; G06T 7/11

(52) U.S. Cl. CPC G06T 7/0016; A61B 3/1225; G06T 7/11; G06T 2207/10024; G06T 2207/20076; G06T 2207/20081; G06T 2207/20084; G06T 2207/30041; G06T 2207/30101

(57) ABSTRACT

Deep learning based systems and methods for predicting and stratifying the risk of glaucoma onset and progression based on color fundus photographs (CFPs) are disclosed. The methods are clinically validated by external population cohorts wherein to apply a machine-learning classifier having been trained using a dataset of CFPs of a longitudinal patient cohort regarding glaucoma development of each of the patients in the cohort over a period of time (e.g., over the course of a few vears), to predict a likelihood of glaucoma incidence or progression for the patient in the future (e.g., over a similar period of time of several years).

### BACKGROUND

Glaucoma is a major chronic eye disease characterized by optic nerve damage and visual field defects (1, 2). Its onset is often insidious, with a risk of irreversible visual field loss prior to becoming symptomatic (3). Timely detection and treatment of glaucoma by lowering the intraocular pressure (IOP) could reduce the risk of disease progression (4, 5). Predicting glaucoma onset and progression is a major clinical challenge. Previous studies demonstrated that biometric parameters, such as baseline IOP, vertical cup-to-disc ratio, mean deviation (in the Humphrey visual field test), and pattern standard deviation, are helpful in predicting glaucoma incidence and progression (6-12). However, IOP measurement and visual field tests are often not available in the primary healthcare setting. In contrast, color fundus photograph (CFP) is widely available and rapid to acquire with the potential to allow artificial intelligence (AI)-based diagnosis of the optic nerve, retinal, and systemic diseases (including chronic kidney disease, diabetes mellitus) (13). Smartphones can also be adapted to capture CFPs, making them a promising tool in disease screening in the future (14, 15). Thus, it would be advantageous if glaucoma incidence and progression could be solely based on CFPs rather than relying on multiple test modalities.

### SUMMARY

In one aspect, a computer-implemented method is provided, comprising using at least one computer processor to receive one or more color fundus photographs (CFPs) of a patient, and apply a machine-learning classifier having been trained using a dataset of CFPs of a patient cohort that have been classified as having glaucoma, to classify the received CFPs of the patient to thereby diagnose whether the patient has glaucoma.

In another aspect, a computer-implemented method is provided, comprising using at least one computer processor to: receive one or more color fundus photographs (CPPs) of a patient; and apply a machine-learning classifier having been trained using a dataset of CFPs of a longitudinal patient cohort regarding glaucoma development of each of the patients in the cohort over a period of time (e.g., over the course of a few years), to predict a likelihood of glaucoma incidence or progression for the patient in the future (e.g., over a similar period of time of several years). The method can be combined with the forgoing method to determine whether the patient currently has glaucoma as well as predict if the patient will develop glaucoma in the future, or the patient's existing glaucoma will progress in the future.

In some embodiments of the methods, the machine-learning classifier comprises a segmentation module based on segmentation of anatomical structures including retinal vessels, macula, optic cup and optic disk from the received CFPs.

In some embodiments, the segmentation module has been trained by manual annotations or segmentations of the anatomical structures including retinal vessels, macula, optic cup and optic disk independently.

In some embodiments, the received one or more CFPs of the patient is obtained from a fundus image of the patient captured by a smart phone.

In some embodiments, the dataset of CFPs the longitudinal patient cohort has been stratified into low-risk and high-risk groups in glaucoma incidence of progression.

In some embodiments, the methods also include using at least one computer processor to classify the patient as belonging to a low-risk or a high-risk group for glaucoma incidence or progression in the future.

In some embodiments, the machine-learning classifier comprises a deep learning model, which may include an architecture of convolutional neural networks (CNN).

### BRIEF DESCRIPTION OF THE DRAWINGS

FIG. 1. Development and validation of the deep learning system for glaucoma diagnosis, glaucoma incidence and progression prediction. A. Data collection and ground truth labelling of glaucoma diagnosis based on CFPs; B. Pipeline for glaucoma diagnosis; C. Data collection and ground truth labelling of glaucoma incidence and progression; D. Pipeline for predicting glaucoma development and progression. CFP, color fundus photo; VF, visual field.

FIG. 2 Area under the receiver operating characteristic (AUROC) curves of the AI model on prediction of glaucoma onset. A to C: predictive performance of the AI model in the validation set (n=1191), external test set 1(n=955), and external test set 2 (n=719).

FIG. 3. Area under the receiver operating characteristic (AUROC) curves of the AI model on prediction of glaucoma progression. A to C: predictive performance of the AI model in the validation set (n=422), external test set 1 (n=337), and external test set 2 (n=513).

FIG. 4. Saliency maps of the deep learning models. Visual explanation of the key regions the models used on diagnostic predictions. a and b: heatmaps of the typical samples of eyes with (a) and without (b) glaucoma development; c and d: heatmaps of the typical samples of eyes with (c) and without (d) glaucoma progression. In both tasks, the saliency maps suggest that the AI model focused on the optic disc rim and areas along the superior and inferior vascular arcades, which are consistent with the clinical approach whereby nerve fiber loss at the superior or inferior disc rim provide key diagnostic clues. AI-based predictions also appear to involve the retinal arterioles and venules.

FIG. 5. Detailed architecture of PredictNet. PredictNet is composed of image preprocessing and analyzing modules. First, in preprocessing stage, the original fundus images are enhanced with Contrast Limited Adaptive Histogram Equalization (CLAHE) and color normalization (NORM). Important retinal structures, including optic disc, optic cup, macula and blood vessels are semantically segmented with trained Unet. The multi-channel anatomical masks output from the Unet are merged into a one-channel mask and then fused with the green and red channels of CLAHE images to form CLAHE Normalization Attention-based images. NORM images are fused with the green and red channels of the original images to form Anatomical Attention-based Images. Second, in analyzing stage, CLAHE Normalization Attention-based images and Anatomical Attention-based Images are fed into two convolutional neural networks, namely ConvNet based model 1 and 2. The final prediction is obtained by integrating the two ConvNet based models in a linear combination.

FIG. 6. Representative samples of automatic segmentation of optic disc, optic cup, macula and blood vessels. a to d: segmentation of optic disc, optic cup, macula and blood vessels. From left to right: original images, manual segmentations, automatic segmentations

FIG. 7. Confusion matrices showing the predictive accuracy of the model across the datasets in the prediction of glaucoma onset. a to c: predictive accuracy in the validation set, and external test set 1, 2. 0 and 1 are labels for eyes without and with glaucoma incidence, respectively.

FIG. 8. Kaplan-Meier curves for predicting glaucoma development accuracy. a to c: predictive accuracy in the validation set, and external test set 1, 2. Survival curves in blue and green represent the high-risk and low-risk subgroups stratified by the upper quartile. P value is computed using a one-sided log-rank test between the two subgroups, and all P values are less than 0.001.

FIG. 9. The distribution of risk scores of the predictive models across all datasets in the prediction of glaucoma onset.

The black dot line represents the low-high threshold of risk score (0.3561). The red bars represent the proportion of eyes without glaucoma development, while the blue bars represent the proportion of eyes with glaucoma development. a to c: glaucoma onset in the validation set, and external test set 1, 2.

FIG. 10. Confusion matrices showing the predictive accuracy of the model across the datasets in the prediction of glaucoma progression. a to c: predictive accuracy in the validation set, and external test set 1, 2. 0 and 1 are labels for eyes without and wit glaucoma progression, respectively.

FIG. 11. AUC curves of the model based on clinical metadata on prediction of glaucoma progression. a to c: predictive performance of the model in the validation set, and external test set 1, 2.

FIG. 12. Kaplan-Meier curves for predicting glaucoma progression accuracy. a to c: predictive accuracy in the validation set, and external test set 1, 2. Survival curves in blue and green represent the high-risk and low-risk subgroups stratified by the upper quartile. P value is computed using a one-sided log-rank test between the two subgroups, and all P values are less than 0.001.

FIG. 13. The distribution of risk scores of the predictive models across all datasets in the prediction of glaucoma progression. The black dot line represents the low-high threshold of risk score (2.6352). The red bars represent the proportion of eyes without glaucoma progression, while the blue bars represent the proportion of eyes with glaucoma progression. a to c: glaucoma onset in the validation set, and external test set 1 and 2.

FIG. 14. Saliency maps of the deep learning models to diagnose glaucoma. Visual explanation of the key regions the models used on diagnostic predictions. a and b: the heatmaps of the typical samples of eyes with (a) and without (b) possible glaucoma. The saliency maps suggest that the AI model focused on the optic disc rim and areas along the superior and inferior vascular arcades, which are consistent with the clinical approach whereby nerve fiber loss at the superior or inferior disc rim provide key diagnostic clues.

### DETAILED DESCRIPTION OF EMBODIMENTS OF THE INVENTION

According to some aspects, disclosed herein are diagnostic systems, computing devices, and computer-implemented methods to diagnose glaucoma and predict glaucoma incidence or progression for a patient (e.g., a human individual) based on color fundus photographs of the patient by using a machine learning framework and without using biopsy. In some embodiments, the machine learning framework utilizes deep learning models such as neural networks.

In one aspect, a computer-implemented method is provided, comprising using at least one computer processor to receive one or more color fundus photographs (CFPs) of a patient, and apply a machine-learning classifier having been trained using a dataset of CFPs of a patient cohort that have been classified as having glaucoma, to classify the received CFPs of the patient to thereby diagnose whether the patient has glaucoma.

In another aspect, a computer-implemented method is provided, comprising using at least one computer processor to: receive one or more color fundus photographs (CFPs) of a patient; and apply a machine-learning classifier having been trained using a dataset of CFPs of a longitudinal patient cohort, i.e., dataset of CFPs captured over a period of time (e.g., a plurality of years), over which the patient may develop glaucoma, to predict a likelihood of glaucoma incidence or progression for the patient in the future. The method can be combined with the forgoing method to determine whether the patient currently has glaucoma as well as predict if the patient will develop glaucoma in the future, or the patient's existing glaucoma will progress in the future.

In some embodiments of the methods, the machine-learning classifier comprises a segmentation module based on segmentation of anatomical structures including retinal vessels, macula, optic cup and optic disk from the received CFPs.

In some embodiments, the segmentation module has been trained by manual annotations or segmentations of the anatomical structures including retinal vessels, macula, optic cup and optic disk independently.

In some embodiments, the received one or more CFPs of the patient is obtained from a fundus image of the patient captured by a smart phone.

In some embodiments, the dataset of CFPs the longitudinal patient cohort has been stratified into low-risk and high-risk groups in glaucoma incidence or progression.

In some embodiments, the methods also include using at least one computer processor to classify the patient as belonging to a low-risk or a high-risk group for glaucoma incidence or progression in the future.

In some embodiments, the machine-learning classifier comprises a deep learning model, which may include an architecture of convolutional neural networks (CNN).

In some embodiments, the systems, devices, media, methods and applications described herein include a digital processing device. For example, in some embodiments, the digital processing device is part of a point-of-care device integrating the diagnostic software described herein. In some embodiments, the medical diagnostic device comprises imaging equipment such as imaging hardware (e.g. a camera, such as a camera of a smart phone) for capturing CRFs. The equipment may include optic lens and/or sensors to acquire CRFs at hundreds or thousands of magnification. In some embodiments, the medical imaging device comprises a digital processing device configured to perform the methods described herein. In further embodiments, the digital processing device includes one or more processors (or computer processors) or hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In accordance with the description herein, suitable digital processing devices include, by way of non-limiting examples, server computers, desktop computers, laptop computers, notebook computers, sub-notebook computers, netbook computers, set-top computers, handheld computers, Internet appliances, mobile smartphones, tablet computers, personal digital assistants, video game consoles, and vehicles. Those of skill in the art will recognize that many smartphones are suitable for use in the system described herein.

In some embodiments, the system, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the system, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application

Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In some embodiments, a computer program includes a web application. In light of the disclosure provided herein, those of skill in the art will recognize that a web application, in various embodiments, utilizes one or more software frameworks and one or more database systems.

In some embodiments, the systems, devices, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location

### EXAMPLE

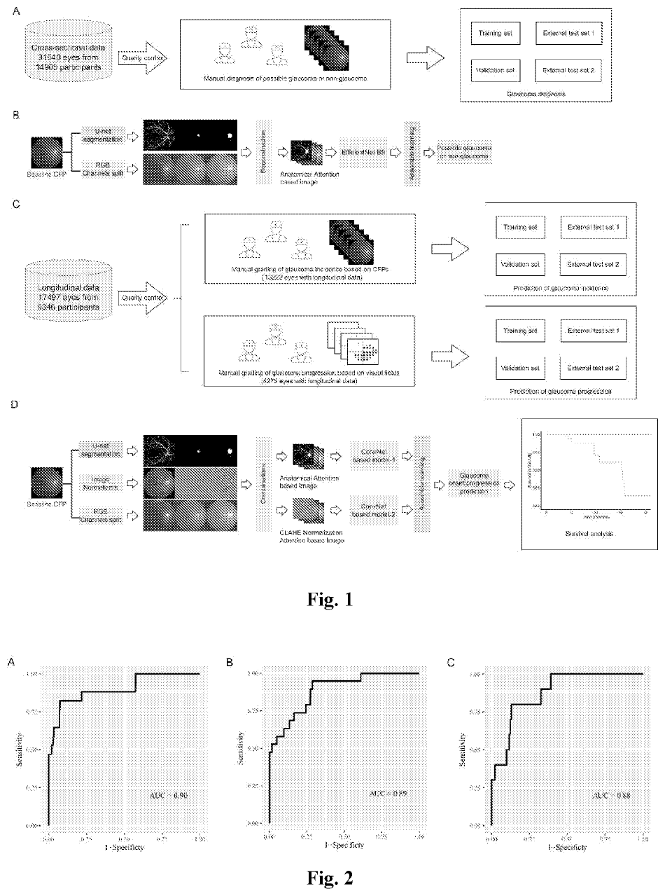

Deep learning techniques have been widely used for glaucoma diagnosis (16-19). However, there is no clinically validated algorithm for glaucoma incidence and progression prediction. This study aims to develop a clinically feasible deep learning system for diagnosing glaucoma (FIGS. 1A & 1B) and predicting the risk of glaucoma onset and progression (FIGS. 1C & 1D) based on CFPs, with validation of performance in external population cohorts. The disclosed AI system and methods are capable of detecting features in the baseline fundus photographs that are unrecognizable to the human eye and predicting which patients will progress to glaucoma within 5 years. Furthermore, the AI system could be deployed at the point-of-care via smartphone image capture to enable broadly accessible remote glaucoma screening in the future.

### Methods

### Dataset Characteristics

Glaucoma diagnosis cohorts. In these initial cohorts, we were specifically looking for patients visiting ophthalmologists who subspecialize in both glaucoma and anterior segment diseases. The population of patients seen by these ophthalmologists was highly enriched with POAG patients (37, 38). We purposely chose these initial cohorts to ensure that we were able to collect sufficient POAG patients as well as non-glaucomatous control patients (such as cataract patients) who were otherwise appropriately matched for developing an AI-based diagnosis of POAG (Table 1). The training and validation data in glaucoma diagnosis were collected from community cohorts and eye clinics in Guangzhou. To test the generalizability of the AI model, two independent datasets obtained from Beijing and Kashi were used as external test sets. The external test set 1 was collected from patients who underwent an annual health check in Beijing city, while the external test set 2 was obtained by smartphones from local eye clinics in Kashi in Xinjiang Autonomous Region.

Glaucoma incidence prediction cohorts. The training and validation data in the prediction of glaucoma incidence were collected from community cohorts in Guangzhou. To test the generalizability of the AI model, two independent datasets obtained from Beijing and Guangzhou communities were used as external test sets. Our longitudinal cohorts for POAG incident prediction have POAG frequencies among 1%-2%, which is well within the norm of the prevalence of POAG in the general population. Glaucoma progression prediction cohorts. The training and validation data in predicting glaucoma progression were collected from one POAG cohort in Zhongshan Ophthalmic Center, Guangzhou. To test the generalizability of the AI model, two independent cohorts composed of PACG and POAG eyes from Zhongshan Ophthalmic Center were used as external test sets.

### Image Quality Control and Labeling

All the images were first de-identified to remove any patient-related information. Fifteen ophthalmologists with at least 10 years of clinical experience were recruited to label the CFPs. First, they were asked to exclude the images with poor quality. The criteria include: 1) optic disc or macula was not fully visible; 2) blurred images due to refractive media. 7.1% of the CFPs were excluded due to poor quality. Second, the graders were asked to assign glaucoma or non-glaucoma labels to each CFP. Third, each glaucomatous eye with longitudinal follow-up data was further analyzed to determine if there is a progression based on the visual field reports during follow-up visits. Visual fields with fixation loss lower than 20%, a false positive rate lower than 15%, and a false negative rate lower than 33% were included. Each CFP or visual field report was evaluated by three ophthalmologists independently and the ground truths were determined by the consensus of three ophthalmologists.

### Criteria of Glaucoma Diagnosis and Progression

Glaucoma was diagnosed using the criteria in previous population-based studies (20-22). Glaucomatous optic neuropathy was defined with the presence of vertical cup-to-disc ratio ≥ 0.7, RNFL defect, rim width ≤0.1 disc diameter, and/or disc hemorrhage. An eye would be labeled as possible glaucoma if one of the above criteria is met.

Glaucoma progression was determined based on the changes in the visual fields (23). The Humphrey Field Analyzer was used to perform all the visual field tests in 24-2 standard mode (Carl Zeiss Meditec, LaJolla, CA, USA). At least three visual field locations worse than baseline at the 5% levels in two consecutive reliable visual fields, or at least three visual field locations worse than baseline at the 5% levels in two consecutive reliable visual fields, were considered as progression (23). The time of progression was defined as the time from baseline to the first visual field that confirmed progression. Three ophthalmologists examined each visual field report separately to determine progression.

### Manual Segmentation of Anatomical Structures

We randomly selected 2000 CFPs for manual segmentations of anatomical structures, including optic disc, optic cup, macula, and blood vessels. Two ophthalmologists independently annotated the CFPs at pixel level, and the final standard reference of annotations was determined by the mean of these two independent annotations.

### Model Design of Glaucoma Prediction and Ocular Disease Diagnosis

First, we developed an AI model, DiagnoseNet, to identify CFPs as glaucoma or non-glaucoma. DiagnoseNet is a pipeline made up of modules for segmentation and diagnosis. The fundus images were first semantically segmented in the segmentation module using Unet (39) to produce four anatomical structures: retinal vessels, macula, optic cup and optic disk. The segmentation data were then merged into a one-channel by element-wise bit or operation over the four anatomical structures-focusing attention layers, which took the place of the CFPs' blue channel to form a new CFP image. The diagnostic module's backbone is EfficientNet-B0, with the last fully connected layer replaced by a Dense layer of two output units initialized with a random value, and the other layers' initial weights determined from ImageNet's pre-trained settings (FIG. 1B).

Then, we created a pipeline, PredictNet, to predict glaucoma onset and progression. PredictNet preprocess and analyze the CFP data (FIG. 1). First, in preprocessing stage, the original fundus images are enhanced with Contrast Limited Adaptive Histogram Equalization (CLAHE) and color normalization (NORM). Important retinal structures, including optic disc, optic cup, macula, and blood vessels are semantically segmented with trained Unet. The multi-channel anatomical masks output from the Unet (39) are merged into a one-channel mask and then fused with the green and red channels of CLAHE images to form CLAHE Normalization Attention-based images. NORM images are fused with the green and red channels of the original images to form Anatomical Attention-based Images. Second, in analyzing stage, CLAHE Normalization Attention-based images and Anatomical Attention-based Images are fed into two convolutional neural networks, namely ConvNet based model 1 and 2. Each ConvNet based model consists of a feature extraction network and a classification network module. The feature extraction network consists of 3 convolutional blocks, which are composed of a Convolution2D layer, a Batch Normalization layer, a LeakReLu layer, and a MaxPooling2D layer in series, while the classification network consists of two Dense layers in series. The GlobalMaxPooling2D layer is used to connect between the feature extraction network and the classification network module. The final prediction is obtained by integrating the two ConvNet based models in a linear combination. At the final step, PredictNet will generate a probability (P) of glaucoma incidence or progression between 0 and 1. P was transferred into a z-score with the formula below:

$$z - score = \frac{P - \overline{P}}{standard deviation of P} ,$$

where P stands for the mean P of each dataset. Then, we got the final standard score by adding 1 to all the z-scores, because some of the z-scores were below zero.

The models were developed with Python (version 3.8.6) and TensorFlow (version 2.1.0).

### Interpretation of the AI Model

Gradient-weighted Class Activation Mapping (Grad-CAM) (40) is used to highlight the class-discriminative region in the image for predicting the decision of interest. We created heat maps generated from CFPs, which indicated the key regions for the AI model to classify the CFPs into low-and high-risk groups.

### Statistics

The demographic characteristics of study participants were presented as mean±standard deviation (SD) for continuous data, and frequency (percentage) for categorical variables. The AUC with 95% confidence interval (CI), sensitivity, and specificity were implemented to assess the performance of the algorithms. Sensitivity and specificity were determined by the selected thresholds in the validation sets. The survival curves were constructed for different risk groups, and the significance of differences between groups was tested by log-rank tests. The predictive performance of AI model and metadata model was performed using DeLong's test. All the hypotheses tested were two-sided, and a p-value of less than 0.05 was considered significant. All statistical analyses were performed using R (ver. 4.0).

### Study Approval

Institutional review board and ethics committee approvals were obtained in all locations and all the participants signed a consent form. All the images were uploaded to a Health Insurance Portability and Accountability Act (HIPAA)-compliant cloud server for further grading.

### Results

### Definitions of Glaucoma, its Incidence and Progression

The diagnostic criteria for possible glaucoma based on CFPs were created following published population-based studies: glaucomatous optic neuropathy was defined by the presence of vertical cup-to-disc ratio ≥0.7, retinal nerve fiber layer (RNFL) defect, optic disc rim width ≤0.1 disc diameter and/or disc hemorrhage (20-22). A glaucoma incidence was defined when baseline CFPs were non-glaucomatous but the eye became possible glaucoma during a follow-up period.

Humphrey visual fields performed in a standard 24-2 pattern mode were used for an analysis when glaucoma progression was suspected (23). Glaucomatous progression was defined by at least three visual field test points worse than the baseline at the 5% levels in two consecutive reliable visual field tests or at least three visual field locations worse than the baseline at the 5% levels in two subsequent consecutive reliable visual field tests (23). Time to progression was defined as the time from a baseline to the first visual field test report that confirmed glaucoma progression following the aforementioned criteria. The gold-standard definition of clinical progression was defined by a unanimous agreement of three ophthalmologists who independently assessed each visual field report.

### Image Datasets and Patient Characteristics

We established a large dataset composed of CFPs and visual fields collected in Guangzhou, Beijing, and Kashi. The demographic and clinical information of the study participants is summarized in Table 1. The data were split randomly into mutually exclusive sets for training, validation, and external testing of the AI algorithms.

In the first task, we developed a model to diagnose possible glaucoma based on 31040 CFP images. In this task, 31040 images (split into training 20872; validation 3182; external test 1:6162; external test 2:824) from 14905 individuals were collected from glaucoma and anterior segment disease eye clinics. 32.8% (10175) of the images were diagnosed with possible glaucoma. The training and validation datasets were obtained from individuals from glaucoma and anterior segment disease sections in Zhongshan Ophthalmic Center in Guangzhou, China. The external test set 1 was collected from patients in the glaucoma and anterior segment disease clinic in Jidong Hospital near Beijing. To further test the generalizability of the AI model, we validated its performance on CFPs obtained by smartphones from Kashi.

In the second task, we developed a model to predict future glaucoma incidence based on the data from three longitudinal cohorts. We included a total of 13222 eyes (training: 10357, validation: 1191, external test 1:955, external test 2:719) of 7127 participants, all of which were diagnosed as non-glaucomatous at the baseline. The training and validation datasets were obtained from individuals who underwent an annual health check in Guangzhou, while external test set 1 was from individuals who underwent an annual health check in Beijing and external test set 2 was from a community cohort in Guangzhou, respectively. The mean follow-up duration is 47.8 to 56.6 months across the datasets. The incidence rate of glaucoma is 1.1% to 2.0% across the datasets.

In the third task, we developed a model to predict glaucoma progression based on the CFPs from cohorts with existing glaucoma. In this task, 4275 eyes (training: 3003, validation: 422; external test 1:337; external test 2:513) from 2219 glaucoma patients were included, all of which were already diagnosed with glaucomatous optic neuropathy at the baseline. The training and validation datasets were obtained from one primary open angle glaucoma (POAG) cohort in Zhongshan Ophthalmic Center. To further test the generalizability of the AI model on different subtypes of glaucoma, external test set 1 was collected from another POAG cohort and external test set 2 was collected from a chronic primary angle-closure glaucoma (PACG) cohort in Zhongshan Ophthalmic Center, respectively. The mean follow-up duration is 34.8 to 41.7 months across the datasets. And the proportion of glaucoma progression is 6% to 13.5% across the datasets (Table 1).

### Design of the Diagnostic (DiagnoseNet) and Predictive Algorithm (PredictNet)

First, we developed a diagnostic algorithm for possible glaucoma, the DiagnoseNet (FIG. 1B). In brief, the DiagnoseNet is composed of two main modules, a segmentation module, and a diagnostic module. The CFPs were semantically segmented by the segmentation module with four anatomical structures including retinal vessels, macula, optic cup, and optic disk. The diagnostic module output the probability score of being glaucomatous.

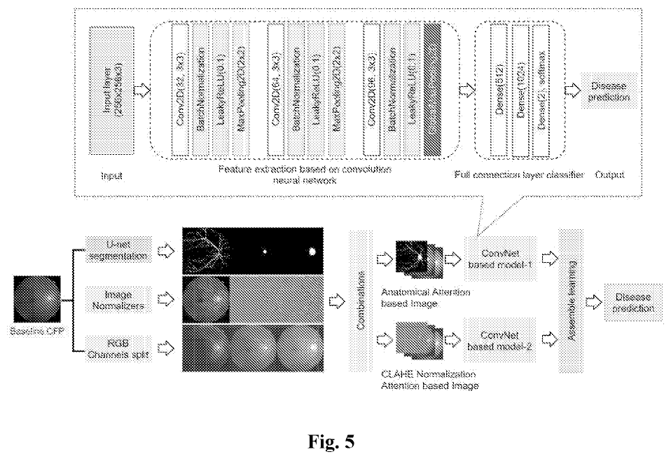

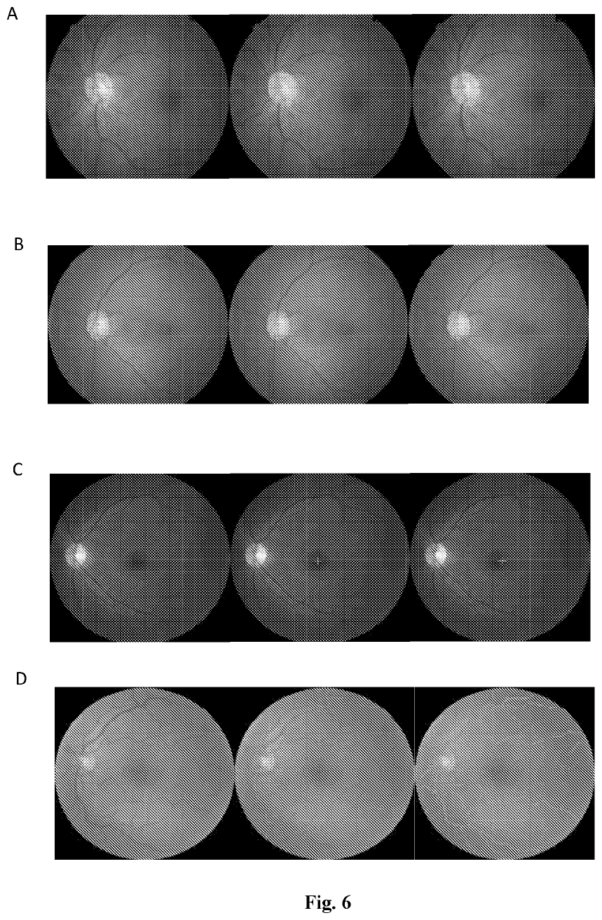

We then designed a pipeline, PredictNet, for incidence and progression prediction of glaucoma. In brief, PredictNet is also composed of two main modules, the segmentation module, and the prediction module. The segmentation module is the same as that in the DiagnoseNet. The prediction module produces the risk score of glaucoma incidence or progression in the future (FIG. 1D & FIG. 5).

The diagnostic and predictive algorithms share the same segmentation module. The segmentation module was trained based on manual annotations of optic disc (1853 images), optic cup (1860 images), macula (1695 images), and blood vessels (160 images) independently. The segmentation module demonstrated outstanding segmentation performance on the above anatomical structures and achieved IOUs of 0.847, 0.669, 0.570, and 0.538 for optic disc, optic cup, macula, and blood vessel segmentation, respectively. Representative samples of segmentation are shown in FIG. 6.

### Diagnostic Performance of the AI Model Based on CFPs Captured by Smartphones

To demonstrate the potential of deploying our AI model in routine healthcare, we developed and tested the AI model to diagnose possible glaucoma based on CFPs not only from fundus cameras but also from smartphones. As shown in Table 2, in this validation dataset, the AI model achieved an AUC of 0.97 (0.96-0.97), a sensitivity of 0.98 (0.97-0.99), and a specificity of 0.82 (0.80-0.83) for differentiating glaucomatous and non-glaucomatous eyes. To evaluate the generalizability of the algorithms, the AI model was tested on two external datasets. In the external test set 1, the AI model achieved an AUC of 0.94 (0.93-0.94), a sensitivity of 0.89 (0.87-0.90), and a specificity of 0.83 (0.81-0.84). In the external test set 2 which was obtained using smartphones, the AI model achieved an AUC of 0.91 (0.89-0.93), a sensitivity of 0.92 (0.88-0.96), and a specificity of 0.71 (0.67-0.74).

### Prediction of Glaucoma Incidence Using Longitudinal Cohorts

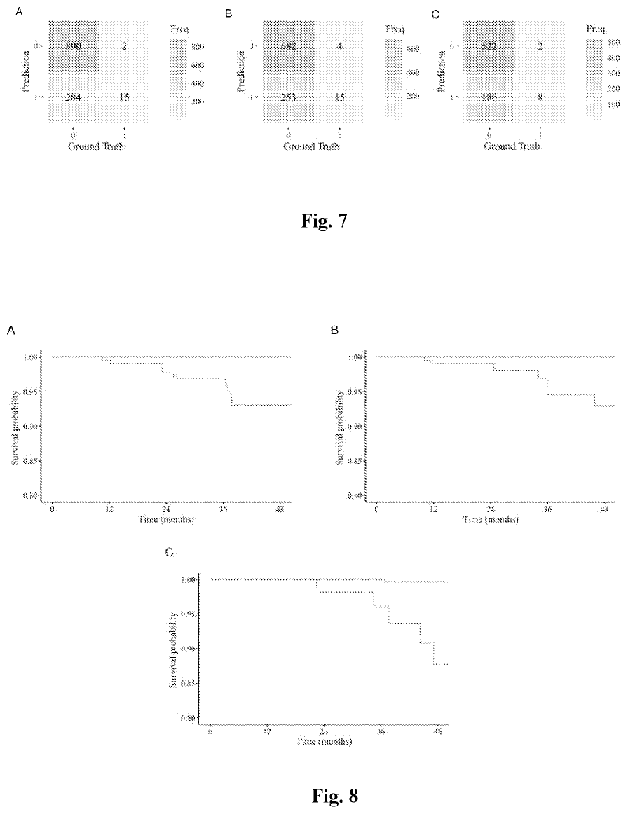

We investigated the predictive performance of the AI model for the development of glaucoma in non-glaucoma individuals over a four to five-year period. A total of 158 eyes developed glaucoma within a four to five-year period. The AI model achieved an AUC of 0.90 (0.81-0.99), a sensitivity of 0.84 (0.82-0.87), and a specificity of 0.82 (0.57-0.96) for predicting glaucoma incidence in the validation set (Table 2 and FIG. 2). The AI model demonstrated good generalizability in the external test sets, which achieved an AUC of 0.89 (0.83-0.95), a sensitivity of 0.84 (0.81-0.86), a specificity of 0.68 (0.43-0.87), and an AUC of 0.88 (0.79-0.97), a sensitivity of 0.84 (0.81-0.86), a specificity of 0.80 (0.44-0.97) in the external test set 1 and 2, respectively (Table 2, FIG. 2, and FIG. 7).

There is a significant difference in the incidence rate of glaucoma between the low-risk and high-risk groups. The incidence rates were 0.2% and 5.0%, 0.6% and 5.6%, 0.4% and 4.1% in the low and high-risk groups of the validation set, external test set 1 and external test set 2, respectively. We employed the Kaplan-Meier approach to stratify healthy individuals into two risk categories (low or high risk) for developing glaucoma, based on four to five-year longitudinal data on glaucoma development. The upper quartile of the predicted risk scores from the model in the validation set was used to create the threshold for the high-risk and low-risk groups in the Kaplan-Meier curves and log-rank tests. In the external test sets, significant separations of the low-and high-risk groups were achieved (both Ps<0.001, FIG. 8).

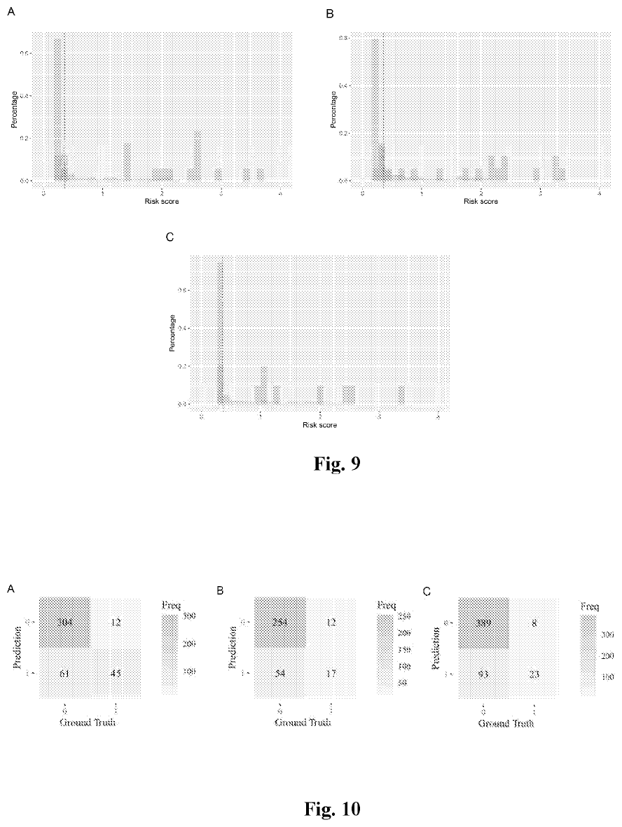

The distribution of the risk scores and the threshold (upper quartile) of low-and high-risk groups across the validation and external test sets are presented in FIG. 9. As shown in the figure, the threshold (risk score of 0.3561, red dot line) well defines a boundary to separate individuals who are likely and unlikely to develop glaucoma in a four to five-year period.

The AI model demonstrated no statistically significant difference in performance among the subgroups as stratified by age (>60 vs <60 years), sex (male vs female), and severity of glaucoma (mean deviation >−6 dB vs <−6 dB).

### Prediction of the Glaucoma Progression Using Longitudinal Chorts

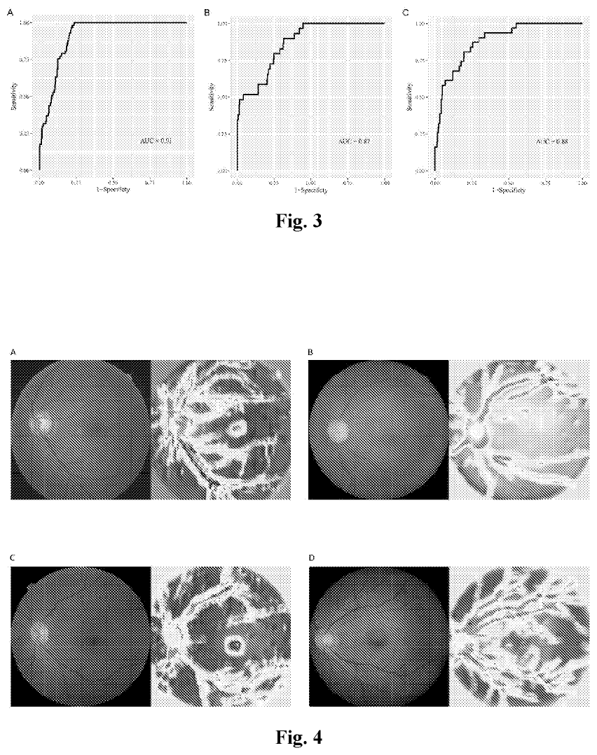

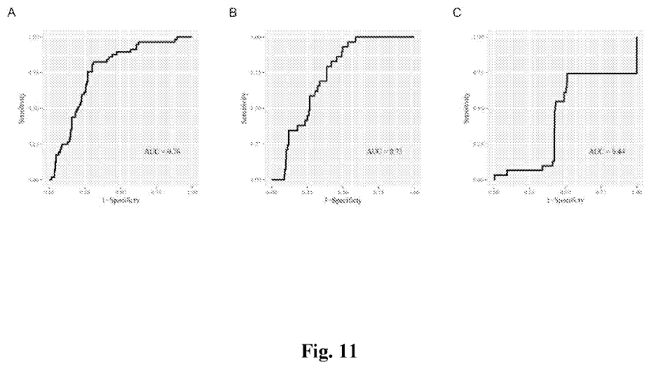

We investigated the predictive performance of the AI model for glaucoma progression in glaucomatous eyes over a three to four-year period. A total of 444 POAG eyes had progression within a three to four-year period. The AI model achieved an AUC of 0.91 (0.88-0.94), a sensitivity of 0.83 (0.79-0.87) and a specificity of 0.79 (0.66-0.89) of for predicting glaucoma progression in the validation set (Table 2 and FIG. 3). To validate the generalizability of the AI model in predicting progression in multi-mechanism glaucoma, we further tested its predictive performance in two independent cohorts of PACG (external test set 1) and POAG (external test set 2). The AI model achieved excellent predictive performance with an AUC of 0.87 (0.81-0.92), a sensitivity of 0.82 (0.78-0.87), a specificity of 0.59 (0.39-0.76), and an AUC of 0.88 (0.83-0.94), a sensitivity of 0.81 (0.77-0.84), a specificity of 0.74 (0.55-0.88) in external test set 1 and 2, respectively (Table 2, FIG. 3 and FIG. 10).

We also trained a predictive model using baseline clinical metadata (age, sex, intraocular pressure, mean deviation, pattern standard deviation, hypertension or diabetes status) alone to predict progression, which led to an AUC of 0.76 (0.70-0.82), 0.73 (0.66-0.79), 0.44 (0.33-0.54) in the validation set, external test set 1 and external test set 2, respectively (FIG. 11). The performance of the AI model is significantly better than that of the predictive model based on baseline metadata in the above datasets (all Ps<0.001).

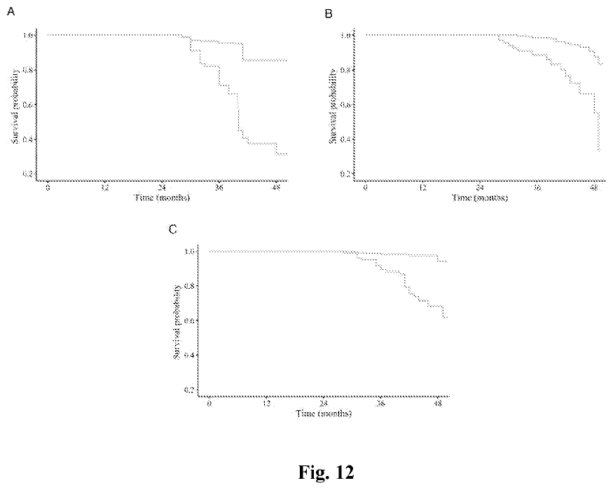

There is a significant difference in the proportion of eyes with glaucoma progression in the low-risk and high-risk groups. The incidence rates were 3.8% and 42.4%, 4.5% and 23.9%, 2.0% and 19.8% in the low and high-risk groups of the validation set, external test set 1 and external test set 2, respectively. We then performed Kaplan-Meier analysis to stratify glaucomatous eyes into two risk categories (low or high risk) for glaucoma progression, based on three to four-year longitudinal data on glaucoma progression. The upper quartile of the predicted risk scores from the model in the validation set was used to create the threshold for the high-risk and low-risk groups in the Kaplan-Meier curves and log-rank tests. In the external test sets, significant separations of the low- and high-risk groups were achieved (both Ps<0.001, FIG. 12).

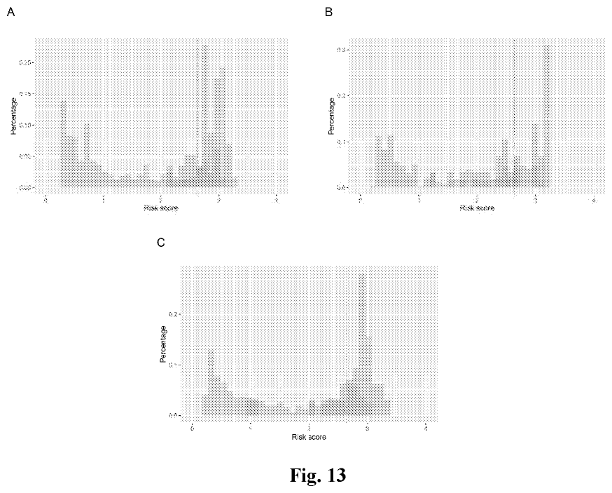

The distribution of the risk scores and the threshold (upper quartile) of low-and high-risk groups across the validation and external test sets are presented in FIG. 13. As shown in the figure, the threshold (risk score of 2.6352, red dot line) well defines a boundary to separate glaucomatous eyes that are likely and unlikely to progress in a three to four-year period.

The AI model demonstrated no statistical significance in all the subgroups stratified by age (≥60 vs <60 years), sex (male vs female), and severity of glaucoma (mean deviation>−age 6 dB vs <−6 dB) except the AUCs of severe and less severe subgroups in the validation and external test set 1.

Visualization of the evidence for prediction of glaucoma incidence and progression

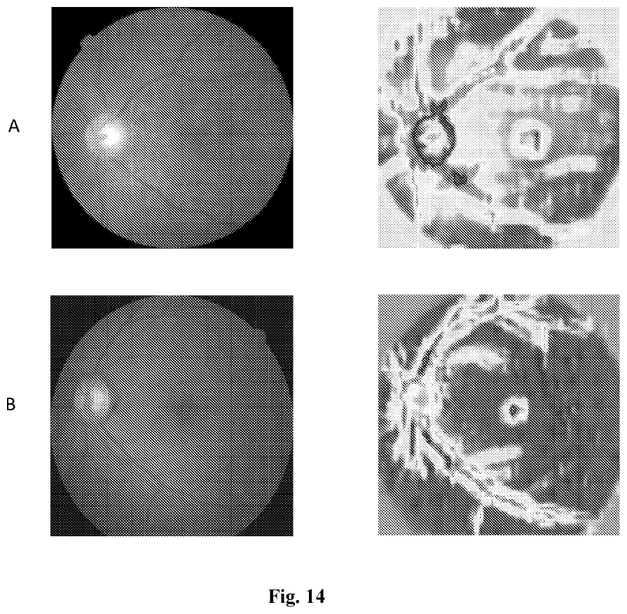

To improve the interpretability of the AI models and illustrate the key regions for AI-based predictions, we used Gradient-weighted Class Activation Mapping (Grad-CAM) to generate the key regions in the CFPs for diagnosing glaucoma, predicting glaucoma incidence and progression. Representative cases and their corresponding saliency maps of DiagnoseNet are presented in FIG. 14. Representative cases and their corresponding saliency maps are presented in FIG. 14 (DiagnoseNet) and FIG. 4 (PredictNet), respectively. The saliency maps suggest that the AI model focused on the optic disc rim and areas along the superior and inferior vascular arcades, which are consistent with the clinical approach whereby nerve fiber loss at the superior or inferior disc rim provides key diagnostic or predictive clues. This would suggest that the AI models are learning clinically relevant knowledge in evaluating glaucoma diagnosis and progression. AI-based predictions also appear to involve the retinal arterioles and venules, thus implicating vascular health as potentially relevant to the etiology of chronic open angle glaucoma.

### Discussion

More than 60 million people in the world suffer from glaucoma, and the number is predicted to increase to 110 million by 2040 (24). Due to its insidious onset and variable progression, diagnosis of glaucoma and monitoring of treatment can be challenging and clinically time-consuming. Glaucoma screening is not universal around the world, thus leading to a delayed diagnosis and severe irreversible sight loss. Therefore, there is a high clinical demand for an efficient and reliable AI model to help identifying high-risk individuals for glaucoma development and progression within the population in order to facilitate early intervention.

Deep learning algorithms have been widely used in glaucoma diagnostic studies (16-19), and have achieved outstanding diagnostic performance in detecting glaucomatous eyes. However, few studies have explored the efficacy of deep learning in glaucoma onset and progression prediction (25-29). In this study, our AI model showed excellent glaucoma diagnostic performance on CFPs, including photographs captured with smartphone cameras using an adaptor which could significantly broaden its application at a point-of-care setting. Compared with traditional statistical models (30-34), such as

Glaucoma Probability Score and Moorfields regression analysis, several studies using deep learning models achieved comparable or even better predictive performance (25-27). Thakur et al. developed AI models to predict glaucoma development approximately 1 to 3 years before clinical onset and achieved a highest AUC of 0.88. However, these deep learning models had some limitations. First, the application was limited to onset prediction without progression prediction, the latter being an essential part of glaucoma management. Second, the data mostly came from hospitals or clinical trials rather than community populations, including many eyes that were diagnosed with ocular hypertension (elevated intraocular pressure without optic neuropathy) rather than glaucoma (25). Third, there is a lack of external validation data to demonstrate the generalizability of the model in the community.

Compared with previous studies, our study has the following advantages. First, we developed AI models for glaucoma diagnosis, incidence and progression prediction. In the external test sets, the models achieved excellent predictive performance in identifying high-risk individuals of developing glaucoma or having glaucoma progression. Secondly, data in glaucoma incidence prediction came from community screening settings, which better reflects the distribution characteristics of glaucoma in the population and facilitates the generalizability of the model. The results in the external datasets show the AI model achieved an excellent predictive performance of glaucoma development, demonstrating strong generalizability and reliability of the AI model. Third, all the patients in the glaucoma cohorts of the progression prediction task have received IOP-lowering medications since enrollment and their IOP values were all controlled within a normal range. This indicates that our predictive model could identify high-risk patients who will undergo glaucoma progression even with reasonably controlled IOPs and facilitate timely interventions such as anti-glaucoma surgeries to save vision. Fourth, the AI model based on structural data from CFPs achieved a high predictive accuracy of glaucoma progression as determined by the gold standard of visual field test results. Visual field tests can reveal functional damage of optic nerve and are the clinical gold standard in monitoring glaucoma progression (35). As demonstrated in the task of glaucoma progression prediction, the AI model succeeded in identifying the high-risk eyes of progressive functional deterioration from baseline CFPs with high sensitivities. In addition, the AI model showed a similar predictive performance in different subtypes of glaucoma, including POAG and PACG, which share similar structural and functional damage of the optic nerve.

Our study has the following limitations. First, the input data of our AI models are only CFPs. Clinical glaucoma evaluation generally requires integrated analysis of multiple modalities (e.g. clinical examination, optic nerve head imaging, and visual field testing) to determine the glaucoma subtypes and any progression. Our study chose CPFs as the only input due to their high feasibility and wide spread availability. Future studies may consider incorporating other data modalities to further improve the predictive performance of the algorithms. Second, only high-quality CFPs were included in the study, which limits the application of the AI models in the eyes with media opacities that exclude obtaining clear CFPs. Third, limited by the prevalence of glaucoma in the general population (around 1% to 1.5% in those aged 40 to 65 years old) (36), there was relatively small number of cases of glaucoma. To address this issue, we used a deep learning model with relatively few parameters. Fourth, the AI models presented varied sensitivity and specificity across the datasets, though having high AUC values. A high sensitivity is more important for screening, and we may further improve the predictive performance of the AI models with more training data in the future. Fifth, all the data were from the Chinese population and further validation is needed in other populations.

In conclusion, our study demonstrates the feasibility of deep learning systems for disease onset and progression prediction. It offers the possibility of building a virtual glaucoma screening system.

### REFERENCES

1. Jonas J B, Aung T, Bourne R R, Bron A M, Ritch R, and Panda-Jonas S. Glaucoma. Lancet. 2017;390(10108):2183-93.
2. Zhang K, Zhang L, and Weinreb R N. Ophthalmic drug discovery: novel targets and mechanisms for retinal diseases and glaucoma. Nat Rev Drug Discov. 2012;11(7):541-59.
3. Weinreb RN, Aung T, and Medeiros F A. The Pathophysiology and Treatment of Glaucoma: A Review. JAMA. 2014;311(18):1901-11.
4. Heijl A, Leske M C, Bengtsson B, Hyman L, Bengtsson B, Hussein M, et al. Reduction of Intraocular Pressure and Glaucoma Progression: Results From the Early Manifest Glaucoma Trial. Archives of Ophthalmology. 2002; 120(10):1268-79.
5. Jammal A A, Thompson A C, Mariottoni E B, Estrela T, Shigueoka L S, Berchuck S I, et al. Impact of Intraocular Pressure Control on Rates of Retinal Nerve Fiber Layer Loss in a Large Clinical Population.Ophthalmology. 2021;128(1): 48-57.
6. Gordon M O, Gao F, Huccker J B, Miller J P, Margolis M, Kass M A, et al. Evaluation of a Primary Open-Angle Glaucoma Prediction Model Using Long-term Intraocular Pressure Variability Data: A Secondary Analysis of 2 Randomized Clinical Trials. JAMA Ophthalmology. 2020;138(7):780-8.
7. Medeiros F A, Alencar L M, Zangwill L M, Bowd C, Sample P A, and Weinreb R N. Prediction of functional loss in glaucoma from progressive optic disc damage. Archives of ophthalmology (Chicago, Ill: 1960). 2009;127(10):1250-6.
8. Coleman A L, Gordon M O, Beiser J A, and Kass M A. Baseline risk factors for the development of primary open-angle glaucoma in the Ocular Hypertension Treatment Study. American Journal of Ophthalmology. 2004;138(4):684-5.
9. Validated Prediction Model for the Development of Primary Open-Angle Glaucoma in Individuals with Ocular Hypertension. Ophthalmology. 2007;114(1):10-9.e2.
10. Song Y, Ishikawa H, Wu M, Liu Y-Y, Lucy K A, Lavinsky F, et al. Clinical Prediction Performance of Glaucoma Progression Using a 2-Dimensional Continuous-Time Hidden Markov Model with Structural and Functional Measurements. Ophthalmology. 2018;125(9):1354-61.
11. Daneshvar R, Yarmohammadi A, Alizadeh R, Henry S, Law S K, Caprioli J, et al. Prediction of Glaucoma Progression with Structural Parameters: Comparison of Optical Coherence Tomography and Clinical Disc Parameters. Am J Ophthalmol. 2019;208:19-29.
12. De Moraes C G, Sehi M, Greenfield D S, Chung Y S, Ritch R, and Liebmann J M. A Validated Risk Calculator to Assess Risk and Rate of Visual Field Progression in Treated Glaucoma Patients. Investigative Ophthalmology & Visual Science. 2012;53(6):2702-7.
13. Zhang K, Liu X. Xu J, Yuan J, Cai W, Chen T, et al. Deep-learning models for the detection and incidence prediction of chronic kidney disease and type 2 diabetes from retinal fundus images. Nature Biomedical Engineering. 2021;5(6):533-45.
14. Wintergerst M W M, Jansen L G, Holz F G, and Finger R P. Smartphone-Based Fundus Imaging-Where Are We Now? The Asia-Pacific Journal of Ophthalmology. 2020;9(4).
15. Iqbal U. Smartphone fundus photography: a narrative review. International Journal of Retina and Vitreous. 2021;7(1):44.
16. Li Z, He Y, Keel S, Meng W, Chang R T, and He M. Efficacy of a Deep Learning System for Detecting Glaucomatous Optic Neuropathy Based on Color Fundus Photographs. Ophthalmology. 2018;125(8):1199-206.
17. Xiong J, Li F, Song D, Tang G, He J, Gao K, et al. Multimodal Machine Learning Using Visual Fields and Peripapillary Circular OCT Scans in Detection of Glaucomatous Optic Neuropathy. Ophthalmology. 2021.

18. Li F, Song D, Chen H, Xiong J, Li X, Zhong H, et al. Development and clinical deployment of a smartphone-based visual field deep learning system for glaucoma detection. npj Digital Medicine. 2020;3(1):123.
19. Li F, Yang Y, Sun X, Qiu Z, Zhang S, Tun T A, et al. Digital Gonioscopy Based on Three-dimensional Anterior-Segment OCT: An International Multicenter Study. Ophthalmology. 2021.
20. Iwase A, Suzuki Y, Araie M, Yamamoto T, Abe H, Shirato S, et al. The prevalence of primary open-angle glaucoma in Japanese: the Tajimi Study. Ophthalmology. 2004;111(9):1641-8.
21. He M, Foster P J, Ge J, Huang W, Zheng Y, Friedman D S, et al. Prevalence and clinical characteristics of glaucoma in adult Chinese: a population-based study in Liwan District, Guangzhou. Invest Ophthalmol Vis Sci. 2006;37(7):2782-8.
22. Foster P J, Buhrmann R, Quigley H A, and Johnson G J. The definition and classification of glaucoma in prevalence surveys. Br J Ophthalmol. 2002;86(2):238-42.
23. Garway-Heath D F, Crabb D P, Bunce C, Lascaratos G, Amalfitano F, Anand N, et al. Latanoprost for open-angle glaucoma (UKGTS): a randomised, multicentre, placebo-controlled trial. Lancet. 2015;385(9975):1295-304.
24. Tham Y C, Li X, Wong T Y, Quigley H A, Aung T, and Cheng C Y. Global prevalence of glaucoma and projections of glaucoma burden through 2040: a systematic review and meta-analysis. Ophthalmology. 2014;121(11):2081-90.
25. Thakur A, Goldbaum M, and Yousefi S. Predicting Glaucoma before Onset Using Deep Learning. Ophthalmology Glaucoma. 2020;3(4):262-8.
26. Thakur A, Goldbaum M, and Yousefi S. Convex Representations Using Deep Archetypal Analysis for Predicting Glaucoma. IEEE Journal of Translational Engineering in Health and Medicine. 2020;8:1-7.
27. Gupta K, Goldbaum M, and Yousefi S. 2021 IEEE/CVF Conference on Computer Vision and Pattern Recognition Workshops (CVPRW). 2021:2259-67.
28. Shuldiner S R, Boland M V, Ramulu P Y, De Moraes C G, Elze T, Myers J, et al. Predicting eyes at risk for rapid glaucoma progression based on an initial visual field test using machine learning. PLoS One. 2021;16(4):e0249856.
29. Wen J C, Lee C S, Keane P A, Xiao S, Rokem A S, Chen P P, et al. Forecasting future Humphrey Visual Fields using deep learning. PLoS One. 2019;14(4):e0214875.
30. Zangwill L M, Weinreb R N, Beiser J A, Berry C C, Cioffi G A, Coleman A L, et al. Baseline topographic optic disc measurements are associated with the development of primary open-angle glaucoma: the Confocal Scanning Laser Ophthalmoscopy Ancillary Study to the Ocular Hypertension Treatment Study. Arch Ophthalmol. 2005;123(9):1188-97.
31. Weinreb R N, Zangwill L M, Jain S, Becerra L M, Dirkes K, Piltz-Seymour J R, et al. Predicting the Onset of Glaucoma: The Confocal Scanning Laser Ophthalmoscopy Ancillary Study to the Ocular Hypertension Treatment Study. Ophthalmology. 2010;117(9):1674-83.
32. Gordon M O, Beiser J A, Brandt J D, Heuer D K, Higginbotham E J, Johnson C A, et al. The Ocular Hypertension Treatment Study: Baseline Factors That Predict the Onset of Primary Open-Angle Glaucoma. Archives of Ophthalmology. 2002;120(6):714-20.
33. Salvetat M L, Zeppieri M, Tosoni C, and Brusini P. Baseline factors predicting the risk of conversion from ocular hypertension to primary open-angle glaucoma during a 10-year follow-up. Eye. 2016;30(6):784-95.
34. Salvetat M L, Zeppieri M, Tosoni C, and Brusini P. Baseline factors predicting the risk of conversion from ocular hypertension to primary open-angle glaucoma during a 10-year follow-up. Eye (Lond). 2016;30(6):784-95.
35. Hu R, Racette L, Chen K S, and Johnson C A. Functional assessment of glaucoma: Uncovering progression. Survey of Ophthalmology. 2020;65(6):639-61.
36. Rudnicka A R, Mt-Isa S, Owen C G, Cook D G, and Ashby D. Variations in Primary Open-Angle Glaucoma Prevalence by Age, Gender, and Race: A Bayesian Meta-Analysis. Investigative Ophthalmology & Visual Science. 2006;47(10):4254-61.
37. Li J. Glaucoma type proportion of glaucoma outpatient in Beijing Tongren Hospital from 2014 to 2016. Investigative Ophthalmology & Visual Science. 2018;59(9):2745- [symbol] .
38. Song Y J, Kim Y W, Park K H, Kim Y K, Choi H J, and Jeoung J W. Comparison of glaucoma patients referred by glaucoma screening versus referral from primary eye clinic. PLOS ONE. 2019;14(1):e0210582.
39. Ronneberger O, Fischer P, and Brox T. In: Navab N, Hornegger J, Wells W M, and Frangi A F eds. Medical Image Computing and Computer-Assisted Intervention—MICCAI 2015. Cham: Springer International Publishing; 2015:234-41.
40. Selvaraju R R, Cogswell M, Das A, Vedantam R, Parikh D, and Batra D. Proceedings of the IEEE international conference on computer vision. 2017:618-26.

TABLE 1
Baseline characteristics of the study participants in the different datasets

| | Training set | | Validation set | | External test set 1 | | External test set 2 | | P₁* | P₂^{#} |
|---|---|---|---|---|---|---|---|---|---|---|
| Glaucoma diagnosis | | | | | | | | | | |
| Number of | 10466 | | 1418 | | 2866 | | 155 | | — | — |
| participants | | | | | | | | | | |
| Number of eyes | 20872 | | 3182 | | 6162 | | 824 | | — | — |
| Male/Female | 2570/7896 | | 351/1067 | | 732/2134 | | 68/87 | | 0.28 | <0.0001 |
| Age (years) | 54.0 | (17.5) | 54.9 | (16.7) | 61.1 | (21.1) | 68.8 | (15.8) | 0.36 | <0.0001 |
| Hypertension (%) | 604 | (5.8%) | 52 | (3.7%) | 135 | (4.7%) | 101 | (64.7%) | 0.03 | <0.0001 |
| Diabetes (%) | 989 | (9.4%) | 84 | (5.9%) | 197 | (6.9%) | 141 | (90.4%) | <0.001 | <0.0001 |
| Number of | 7034 | (33.7%) | 952 | (29.9%) | 2037 | (33.1%) | 152 | (18.4%) | 0.35 | <0.0001 |
| glaucomatous eyes | | | | | | | | | | |
| (%) | | | | | | | | | | |
| Glaucoma onset | | | | | | | | | | |
| prediction | | | | | | | | | | |
| Number of | 5548 | | 657 | | 522 | | 400 | | — | — |
| participants | | | | | | | | | | |
| Number of eyes | 10357 | | 1191 | | 955 | | 719 | | — | — |
| Male/Female | 2726/2822 | | 321/336 | | 241/281 | | 190/210 | | 0.20 | 0.53 |
| Age (years) | 55.3 | (13.4) | 56.5 | (15.5) | 55.4 | (13.9) | 55.6 | (13.3) | 0.85 | 0.64 |
| Hypertension (%) | 1961 | (35.3%) | 186 | (28.3%) | 184 | (35.2%) | 155 | (38.8%) | 0.97 | 0.17 |
| Diabetes (%) | 3156 | (56.9%) | 285 | (43.4%) | 123 | (23.6%) | 113 | (15.7%) | <0.0001 | <0.0001 |
| Follow-up duration | 47.8 | (15.8) | 56.6 | (13.9) | 51.4 | (11.8) | 52.9 | (13.5) | <0.0001 | <0.0001 |
| (months) | | | | | | | | | | |
| Mean time between | 42.5 | (14.3) | 51.4 | (12.8) | 46.0 | (13.2) | 47.4 | (13.0) | — | — |
| CFPs (months) | | | | | | | | | | |
| Median time | 38.0 | | 56.6 | | 46.0 | | 47.0 | | — | — |
| between CFPs | | | | | | | | | | |
| (months) | | | | | | | | | | |
| Eyes with glaucoma | 112 | (1.1%) | 17 | (1.4%) | 19 | (2.0%) | 10 | (1.4%) | <0.0001 | 0.02 |
| incidence (%) | | | | | | | | | | |

| Glaucoma progression | Training set | | Validation set | | External test set 1 | | External test set 2 | | | |
|---|---|---|---|---|---|---|---|---|---|---|
| prediction | (POAG patients) | | (POAG patients) | | (POAG patients) | | (PACG patients) | | P₁* | P₂^{#} |
| Number of | 1558 | | 217 | | 172 | | 272 | | — | — |
| participants | | | | | | | | | | |
| Number of eyes | 3003 | | 422 | | 337 | | 513 | | — | — |
| Male/Female | 639/919 | | 76/141 | | 76/96 | | 117/155 | | 0.42 | 0.54 |
| Age (years) | 44.7 | (14.3) | 42.8 | (13.2) | 41.7 | (15.2) | 45.1 | (13.9) | 0.008 | 0.71 |
| Intraocular | 16.4 | (3.6) | 16.6 | (3.2) | 16.2 | (3.5) | 16.6 | (3.7) | 0.89 | 0.27 |
| pressure (mmHg) | | | | | | | | | | |
| Mean deviation (dB) | −2.6 | (4.1) | −2.6 | (4.8) | −2.2 | (3.6) | −2.7 | (4.5) | 0.12 | 0.75 |
| Pattern standard | 3.9 | (3.9) | 3.8 | (4.0) | 3.2 | (3.4) | 4.2 | (4.3) | 0.0009 | 0.24 |
| deviation (dB) | | | | | | | | | | |
| Mean times of VF | 7.2 | (3.6) | 6.5 | (2.4) | 7.1 | (3.5) | 6.3 | (2.3) | 0.67 | <0.0001 |
| tests | | | | | | | | | | |
| Mean time between | 6.0 | (2.0) | 6.1 | (1.4) | 6.4 | (2.3) | 7.6 | (1.1) | — | — |
| VF tests (months) | | | | | | | | | | |
| Hypertension (%) | 197 | (12.6%) | 26 | (12.0%) | 31 | (18.0%) | 35 | (12.9%) | 0.05 | 0.92 |
| Diabetes (%) | 8 | (0.5%) | 0 | (0) | 0 | (0%) | 0 | (0%) | 0.35 | 0.24 |
| Follow-up duration | 41.7 | (4.2) | 34.8 | (5.8) | 39.8 | (5.9) | 38.4 | (7.1) | <0.0001 | <0.0001 |
| (months) | | | | | | | | | | |
| Eyes with glaucoma | 327 | (10.9%) | 57 | (13.5%) | 29 | (8.6%) | 31 | (6.0%) | 0.20 | 0.80 |
| progression (%) | | | | | | | | | | |

VF, visual field; POAG, primary open angle glaucoma; PACG, primary angle closure glaucoma

*Comparison of the demographic parameters between training and external test dataset 1 by independent t-test (age, follow-up duration, intraocular pressure, mean deviation, pattern standard deviation, and times of visual field tests) or chi-square test (sex, cases with hypertension, cases with diabetes, cases with glaucoma diagnosis/incidene/progression)

#Comparison of the demographic parameters between training and external test dataset 2 by independent t-test (age, follow-up duration, intraocular pressure, mean deviation, pattern standard deviation, and times of visual field tests) or chi-square test (sex, cases with hypertension, cases with diabetes, cases with glaucoma diagnosis/incidene/progression)

TABLE 2
Performance of the deep learning models in the validation and external test sets

| | AUC (95% CI) | Sensitivity (95% CI) | Specificity (95% CI) | Precision (95% CI) |
|---|---|---|---|---|
| Glaucoma diagnosis | | | | |
| Validation set | 0.97 (0.96-0.97) | 0.98 (0.97-0.99) | 0.82 (0.80-0.83) | 0.99 (0.98-0.99) |
| External test set 1 | 0.94 (0.93-0.94) | 0.89 (0.87-0.90) | 0.83 (0.81-0.84) | 0.94 (0.93-0.94) |
| External test set 2 | 0.91 (0.89-0.93) | 0.92 (0.88-0.96) | 0.71 (0.67-0.74) | 0.97 (0.95-0.99) |
| Glaucoma incidence | | | | |
| prediction | | | | |
| Validation set | 0.90 (0.81-0.99) | 0.84 (0.82-0.87) | 0.82 (0.57-0.96) | 1.00 (0.99-1.00) |
| External test set 1 | 0.89 (0.83-0.95) | 0.84 (0.81-0.86) | 0.68 (0.43-0.87) | 0.99 (0.98-1.00) |
| External test set 2 | 0.88 (0.79-0.97) | 0.84 (0.81-0.86) | 0.80 (0.44-0.97) | 1.00 (0.99-1.00) |
| Glaucoma progression | | | | |
| prediction | | | | |
| Validation set | 0.91 (0.88-0.94) | 0.83 (0.79-0.87) | 0.79 (0.66-0.89) | 0.96 (0.93-0.98) |
| External test set 1 | 0.87 (0.81-0.92) | 0.82 (0.78-0.87) | 0.59 (0.39-0.76) | 0.95 (0.92-0.98) |
| External test set 2 | 0.88 (0.83-0.94) | 0.81 (0.77-0.84) | 0.74 (0.55-0.88) | 0.98 (0.96-0.99) |

1. A method comprising using at least one computer processor to:
receive one or more color fundus photographs (CFPs) of a patient;
apply a machine-learning classifier having been trained using a dataset of CFPs of a patient cohort that have been classified according to their glaucoma status, to classify the received CFPs of the patient to thereby diagnose whether the patient has glaucoma.

2. A method comprising using at least one computer processor to:
receive one or more color fundus photographs (CFPs) of a patient;
apply a machine-learning classifier having been trained using a dataset of CFPs of a longitudinal patient cohort regarding glaucoma development of each of the patients in the cohort over a period of time, to predict a likelihood of glaucoma incidence or progression for the patient in the future.

3. The method of claim 1, wherein the machine-learning classifier comprises a segmentation module based on segmentation of anatomical structures including retinal vessels, macula, optic cup and optic disk from the received CFPs.

4. The method of claim 3, where the machine-learning classifier further comprises a diagnostic module which generates a glaucomatous probability score.

5. The method of claim 2, wherein the machine-learning classifier comprises a segmentation module based on segmentation of anatomical structures including retinal vessels, macula, optic cup and optic disk from the received CFPs.

6. The method of claim 5, wherein the machine-learning classifier further comprises a prediction module which produces a risk score of glaucoma incidence or progression in the future for the patient.

7. The method of claim 3, wherein the segmentation module has been trained by manual annotations or segmentations of the anatomical structures including retinal vessels, macula, optic cup and optic disk independently.

8. The method of claim 1, wherein the received one or more CFPs of the patient is obtained from a fundus image of the patient captured by a smart phone.

9. The method of claim 2, wherein the dataset of CFPs the longitudinal patient cohort has been stratified into low-risk and high-risk groups in glaucoma incidence or progression.

10. The method of claim 9, further comprising: using at least one computer processor to classify the patient as belonging to a low-risk or a high-risk group for glaucoma incidence or progression in the future.

11. The method of any of the foregoing claims claim 1, wherein the machine-learning classifier comprises a deep learning model.

12. The method of claim 11, wherein the deep learning model comprises convolutional neural networks (CNN).

13. The method of claim 1, wherein the machine-learning classifier comprises segmenting the anatomical structures including retinal vessels, macula, optic cup and optic disk of the patient's CFPs using a U-net architecture.

14. The method of claim 5, wherein the segmentation module has been trained by manual annotations or segmentations of the anatomical structures including retinal vessels, macula, optic cup and optic disk independently.
